(19) United States
(12) Patent Application Publication
(10) Pub. No.: US 2007/0265275 A1
(43) Pub. Date: Nov. 15, 2007

(54) Treatment of Mastitis

(75) Inventors: Franz Pirro, Langenfeld (DE); Kristine Fraatz, Burscheid (DE); Robrecht Froyman, Monheim (DE)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: BAYER HEALTHCARE AG, D-51368 LEVERKUSEN (DE)

(21) Appl. No.: 11/718,999

(22) PCT Filed: Oct. 28, 2005
(86) PCT No.: PCT/EP05/11553

§ 371 Date: May 10, 2007

(30) Foreign Application Priority Data
Nov. 12, 2004 (DE) 10 2004 054 873.0

(51) Int. Cl. A61K 31/496; A61P 29/00

(52) U.S. Cl. 514/253.08

(57) ABSTRACT

The invention relates to the simplified treatment of mastitis with enrofloxacin or ciprofloxacin, in particular in the case of cows.

The invention relates to the simplified treatment of mastitis with enrofloxacin or ciprofloxacin, in particular in the case of cows.

The active compound enrofloxacin has been successfully employed for years in many countries for treating bacterially determined infectious diseases in animals (Baytril®). While the classical areas of use primarily comprise respiratory and enteric diseases, skin infections, urinary tract infections, teat infections and joint infections are also successfully treated. The customary treatment scheme in this connection envisages repeated administration over a period of from three to five days. Attempts to shorten the period of treatment while retaining the size of the dose have led in the past to the loss of the sought-after therapeutic efficacy.

U.S. Pat. No. 5,756,506 relates to the treatment of infections with a single administration of fluoroquinolones, such as enrofloxacin; however, this treatment uses a markedly higher dose.

It has now been found, surprisingly, that parenterally administered enrofloxacin has an unexpectedly good effect in the treatment of mastites (udder inflammations), such that the number of administrations can be reduced and the treatment thereby simplified.

The invention therefore relates to the use of enrofloxacin for producing pharmaceuticals for the parenteral treatment of bacterially determined mastites with at most two administrations.

The invention furthermore relates to a method for treating bacterially determined mastites, in which method enrofloxacin is parenterally administered at most twice to the animal in question.

Without this thereby limiting the invention, this surprising finding can be explained by the following investigative results: it was already known from serum kinetics investigations that, following administration, a small proportion of the enrofloxacin is metabolized to ciprofloxacin. However, the effect of the enrofloxacin is normally also in fact essentially to be attributed to this molecule and not to its metabolite ciprofloxacin. In connection with investigating the substances having an antibacterial effect in bovine milk following the parenteral administration of enrofloxacin, we discovered a high antibacterial activity (enrichment of active compounds as compared with the serum concentration) in association with a surprisingly high proportion of ciprofloxacin (of the order of size of 90%) and a surprisingly low proportion of enrofloxacin (of the order of size of 10%) in the milk; this is roughly a reversal of the ratio that was expected. In-vitro activity comparisons show that, in the case of bacterial species which play an important role as pathogens in mastites, ciprofloxacin has a markedly more powerful effect than enrofloxacin.

According to another embodiment, the invention therefore relates to the use of ciprofloxacin for producing pharmaceuticals for treating mastitis.

Enrofloxacin is a fluoroquinolonecarboxylic acid having the systematic designation 1-cyclopropyl-7-(4-ethyl-1-piperazinyl)-6-fluoro-1,4-dihydro-4-oxo-3-quinolone-carboxylic acid:

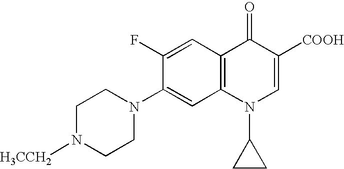

Ciprofloxacin has the systematic designation 1-cyclopropyl-7-(1-piperazinyl)-6-fluoro-1,4-dihydro-4-oxo-3-quinolonecarboxylic acid:

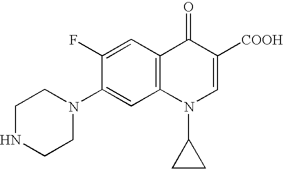

The active compounds can be used in the form of their pharmaceutically acceptable salts, specifically in the form of salts with inorganic acids, such as hydrochloric acid, hydrobromic acid, hydroiodic acid, sulphuric acid or phosphoric acid, or organic acids, such as formic acid, acetic acid, propionic acid, lactic acid, maleic acid, fumaric acid, citric acid, ascorbic acid, succinic acid, glutaric acid and tartaric acid, polyhydroxycarboxylic acids, such as gluconic acid, galacturonic acid and glucuronic acid, amino acids, such as glutamic acid and aspartic acid, and sulphonic acids, such as methanesulphonic acid and ethanesulphonic acid. Suitable bases for forming salts are, for example, inorganic bases, such as NaOH, KOH, Ca(OH)₂ and ammonia, and organic bases, such as amines, e.g. mono-, di- and trialkylamines, substituted amines, such as ethanolamine, cyclic amines, such as morpholine or piperazine, basic amino acids, such as arginine, lysine and codeine, or N-methylglucamine. The active compounds and their preparation are described, for example, in U.S. Pat. No. 4,670,444.

Preparations for the parenteral administration are likewise known in principle, see, for example, U.S. Pat. No. 4,772,605 and U.S. Pat. No. 5,998,418, which publications are hereby expressly incorporated by reference.

Emulsions, suspensions and, in particular, solutions are suitable for the parenteral administration.

The preferred solvent is water, which can, where appropriate, also be used in a mixture with other solvents. These other solvents include: alcohols such as monohydric or polyhydric primary or secondary or tertiary alcohols (e.g. ethanol, butanol, benzyl alcohol, glycol, propylene glycol, triethylene glycol, polyethylene glycol, glycerol and propylene glycol) as well as N-methylpyrrolidone.

However, it is also possible to conceive of oil-based preparations; these are usually suspensions. In the preparations according to the invention, the active compounds are generally present at concentrations of from 0.1 to 30% by weight, preferably from 0.5 to 20% by weight, particularly preferably from 1 to 10% by weight.

The use of highly pure quinolonecarboxylic acids for preparing parenterally administrable solutions is described in EP-A-287 926; this document is hereby expressly incorporated by reference.

Acidic formulations can be used; preferred pH values are in the range from pH 3 to 6.5, particularly preferably from 3 to 5. The acids employed can in principle be those which are mentioned above for forming salts; preferred examples are lactic acid and gluconolactone. Solutions of the lactic acid salts of quinolonecarboxylic acids, in particular ciprofloxacin, which are suitable for injection purposes are described in EP-A-138 018; other acidic infusion solutions of ciprofloxacin are disclosed in EP-A-219 784; acidic injection solutions for enrofloxacin are described in U.S. Pat. No. 5,998,418; these three documents are hereby expressly incorporated by reference.

Preference is given to basic formulations which contain superequimolar quantities of bases; these preparations have a pH of from 8 to 12.5, preferably from 9 to 12, particularly preferably from 9.5 to 11.5. Suitable bases are, for example, those mentioned above in connection with the salts, preferably the alkali metal hydroxides such as NaOH and, in particular, KOH. A base which is also particularly preferred is arginine. These formulations are, for example, described in more detail in U.S. Pat. No. 4,772,605; this document is hereby expressly incorporated by reference.

The pharmaceutical preparations can also comprise customary auxiliary substances; these are nontoxic pharmaceutical substances such as diluents, thickeners, absorption accelerators, absorption inhibitors, crystal growth inhibitors, complexing agents, light-stability agents, antioxidants and preservatives. The following may be mentioned by way of example: as thickeners, methyl cellulose, hydroxyethyl cellulose, hydroxypropyl cellulose, sodium carboxymethyl cellulose, polyvinylpyrrolidone and gelatine; as preservatives, p-hydroxybenzoic acid esters, phenols, chlorobutanol, benzyl alcohol, ethanol, butanol, 1,3-butanediol, chlorohexidine salts, benzoic acid and salts, and sorbic acid; as antioxidants, ascorbic acid, L-cystein, thiodipropionic acid, thiolactic acid, monothioglycerol, propyl gallate, sodium metadisulphite or sodium sulphite; as complexing agents, sodium salts of ethylenediaminetetraacetic acid, phosphates, acetates and citrates; as a crystal growth inhibitor, polyvinylpyrrolidone. Local anaesthetics, such as procaine hydrochloride or lidocaine hydrochloride, can be added where appropriate. The concentration of the auxiliary substances which may possibly be employed varies greatly and, in customary formulations, can be in the range of from 0.1 to 30% by weight for the total quantity of auxiliary substances present.

Sodium chloride, glucose, fructose, glycerol, sorbitol, mannitol, sucrose, xylitol, or mixtures of these substances, can, for example, be added in a quantity which is suitable for establishing isotonic conditions.

In principle, bacterially determined, in particular coliform, mastites can, in accordance with the invention, be treated in all mammals. However, the treatment of milk-yielding productive animals is of particular importance; preferred examples which may be mentioned are: sheep, goats and, in particular, cows. The following pathogens may, in particular, be mentioned in this connection: E. coli, Klebsiella spp., Enterobacter spp., Salmonella spp., Citrobacter spp., Serratia spp., Shigella spp., Edwardsiella spp., Hafnia spp., Morganella spp., Providencia spp., Yersinia spp., Staphylococcus aureus, Staphylococcus spp., Pseudomonas spp., Mycoplasma spp. and Erwinia spp., and the following infections of the mammary gland which are caused by noncoliform bacteria.

Administration is effected parenterally, usually by means of injection, for example intramuscularly, preferably intravenously or subcutaneously.

In the treatment, from 1 to 10 mg, preferably from 2 to 8 mg, particularly preferably from 2.5 to 7 mg, of the active compound per kg of body weight are usually administered per day. The administration is preferably effected on two consecutive days. Only one administration is normally required per day.

In addition, frequently occurring mixed and monoinfections, or mixed infections with, for example, E. coli and staphylococcus or mycoplasma are treated satisfactorily.

### EXAMPLES

### Formulation Examples

The formulations of the following examples can be employed in accordance with the invention. Their preparation is disclosed in the prior art:

### Example 1

100 ml contain:

| | |
|---|---|
| 10.0 g | of enrofloxacin |
| 8.0 g | of gluconolactone |
| 1.40 g | of benzyl alcohol |
| 0.1 g | of sodium sulphite |
| 86.7 g | of water (for injection purposes) |

pH = 3.90

### Example 2

100 ml contain:

| | |
|---|---|
| 5.0 g | of enrofloxacin |
| 3.0 g | of gluconolactone |
| 1.00 g | of benzyl alcohol |
| 0.1 g | of sodium sulphite |
| 93.6 g | of water (for injection purposes) |

pH = 4.40

### Example 3

100 ml contain:

| | |
|---|---|
| 5.0 g | of enrofloxacin |
| 3.0 g | of n-butanol |
| | KOH to pH 11 |
| q.s. | water (for injection purposes) |

### Example 4

100 ml contain:

| | |
|---|---|
| 10.0 g | of enrofloxacin |
| 3.0 g | of n-butanol |
| | KOH to pH 11 |
| q.s. | water (for injection purposes) |

### Example 5

100 ml contain:

| | |
|---|---|
| 10.0 g | of enrofloxacin |
| 3.0 g | of n-butanol |
| 2.0 g | of benzyl alcohol |
| 20.0 g | of L-arginine |
| q.s. | water (for injection purposes) |

### Example 6

100 ml contain:

| | |
|---|---|
| 200 mg | of ciprofloxacin |
| 321.8 mg | of lactic acid solution |
| 900 mg | of NaCl |
| 140 mg | of hydrochloric acid |
| q.s. | water (for injection purposes) |

### Example 7

100 ml contain:

| | |
|---|---|
| 200 mg | of ciprofloxacin |
| 372.5 mg | of 10% (w/w) lactic acid |
| 900 mg | of NaCl |
| 10.4 mg | of hydrochloric acid |
| q.s. | water (for injection purposes) |

pH = 3.7

### Example 8

100 ml contain:

| | |
|---|---|
| 100 mg | of ciprofloxacin |
| 320 mg | of 10% (w/w) lactic acid |
| 625 mg | of NaCl |
| q.s. | water (for injection purposes) |

pH = 4.4

### Example 9

100 ml contain:

| | |
|---|---|
| 42.4 mg | of ciprofloxacin calcium salt |
| 644 mg | of 2% (w/w) lactic acid |
| 5.06 mg | of hydrochloric acid |
| 520 mg | of glycerol |
| q.s. | water (for injection purposes) |

pH = 4.3

### Example 10

100 ml contain:

| | |
|---|---|
| 254.5 mg | of ciprofloxacin × 5H₂O |
| 1284 mg | of 5% (w/w) lactic acid |
| 3.3 mg | of hydrochloric acid |
| 2500 mg | of glucose |
| q.s. | water (for injection purposes) |

pH = 4.2

### Example 11

100 ml contain:

| | | |
|---|---|---|
| 233 | mg | of ciprofloxacin potassium salt |
| 277 | mg | of 20% (w/w) lactic acid |
| 8.86 | ml | of 0.1 M hydrochloric acid |
| 5000 | mg | of glucose |

| | |
|---|---|
| q.s. | water (for injection purposes) |

pH = 4.6

### Biological Example

Clinical Study

In a clinical study, the efficacy of enrofloxacin (Baytril® 10% injection solution, commercial product) in the treatment of mastitis was compared with that of a cefquinome-based commercial product (Cobactan LC®) which is customarily used for treating mastitis. Enrofloxacin was administered intravenously in a dose of 5 mg/kg of body weight once daily on two consecutive days. Cefquinome was administered, by intramammary administration into the infected udder quarter, at a dose of 75 mg every 12 hours after three consecutive milkings.

Result:

Taken overall, the enrofloxacin group was in better condition after the treatment than was the group which was treated with the comparison product.

The treatment with enrofloxacin was well tolerated by all the cows.

The study showed that, with the administration as stated, the enrofloxacin product is suitable for treating acute coliform mastitis in dairy cows. When the general and local symptoms, the milking performance and the bacteriological results were assessed, enrofloxacin was found to be superior to the comparison product.

1. Use of enrofloxacin for producing pharmaceuticals for the parenteral treatment of mastites by means of at most two administrations.

2. Method for treating mastites in which enrofloxacin is administered parenterally at most twice to the animal in question.

3. Use of ciprofloxacin for producing pharmaceuticals for treating mastitis.
